(19) United States
(12) Patent Application Publication
(10) Pub. No.: US 2011/0183380 A1
(43) Pub. Date: Jul. 28, 2011

(54) POROUS, CARBOHYDRATE-BASED FOAM STRUCTURES AND ASSOCIATED METHODS

(75) Inventors: Khaled El-Tahlawy, Raleigh, NC (US); Renzo Shamey, Raleigh, NC (US)

(73) Assignee: NORTH CAROLINA STATE UNIVERSITY, Raleigh, NC (US)

(21) Appl. No.: 13/014,435

(22) Filed: Jan. 26, 2011

Related U.S. Application Data
(60) Provisional application No. 61/298,228, filed on Jan. 26, 2010.

(51) Int. Cl. C08B 30/00; C12P 19/04; C08B 30/04; C08B 30/12; B82Y 30/00; B82Y 40/00

(52) U.S. Cl. 435/101; 127/69; 127/70; 525/54.24; 127/32; 977/773; 977/795; 977/840

(57) ABSTRACT

Porous, carbohydrate-based foam structures and associated methods are disclosed. According to an aspect, a method can include using a starch solution. The starch solution can be precipitated to form starch nanoparticles having a predefined void structure.

### CROSS REFERENCE TO RELATED APPLICATION

This application claims priority to U.S. Provisional Patent Application No. 61/298,228, filed on Jan. 26, 2010, the entire content of which is hereby incorporated by reference herein.

### TECHNICAL FIELD

The presently disclosed subject matter relates to foam structures. Particularly, the presently disclosed subject matter relates to porous, starch-based foam structures and associated methods.

### BACKGROUND

Many consumer and industrial products are formed from non-renewable materials such as petroleum based materials. Much research and investment efforts has been undertaken in order to produce suitable renewable materials for consumer and industrial products.

These efforts have yet to produce a cost-effective material that may be utilized in a variety of settings and has sufficient properties such as strength, thermal and electrical conductivity, and density and that is made from a renewable material source. For example, various foam structures have been produced using petroleum based compounds that may have had sufficient material properties, however, these structures were not made from a renewable material source.

It is desirable to provide alternative methods for producing foam structures without the use of petroleum based compounds. Particularly, it is desirable to produce foam structures based on renewable materials.

### SUMMARY

The presently disclosed subject matter relates to porous, carbohydrate-based foam structures and related methods. Particularly, the presently disclosed subject matter can include nanocellular foam structures based on modified starch from renewable natural polymers. The presently disclosed foam structures have unique properties including, but not limited to, significantly increased specific surface area, high degree of brightness and opacity, light weight and porosity which can be used to design and develop a number of mechanisms for the storage and the gradual release of suitable compounds. Foam structures having predefined features, such as particle size and void structure, can be provided through estimation and assessment of the composition-structure-processing relationships. The novel, porous, starch-based foam structures disclosed herein may be altered to have a hydrophobic character with associated applications in, including but not limited to, medical, electrical and civil engineering, food, agriculture, and packing industries.

The development and use of the presently disclosed novel high performance nanofoam particles from inexpensive, readily available natural starch resources can be expected to lead directly to assisting the engineering, paper, textiles, agricultural, food and pharmaceutical industries. In addition, this sustainable technology can be expected to significantly benefit the environment with associated gains for the consumer, industry and the nation.

In accordance with an aspect of the presently disclosed subject matter, a method for producing a porous, starch-based foam structure is provided. The method can include providing a starch solution, which can be precipitated to form starch nanoparticles with a defined void structure.

According to an aspect, the starch solution can comprise starch slurry of native corn cooked starch or gelatinized starch. Alternative to use of native corn cooked starch as described herein, wheat cooked starch or potato cooked starch may be used. The starch solution can comprise water and starch particles. In one example, the starch particles can comprise about 20 percent of the starch solution.

According to another aspect, the step of precipitating the starch solution can comprise exchanging the water with a solvent having a lower surface tension than the water. The step of exchanging the water with a solvent can be repeated.

According to another aspect, under agitation and prior to the step of precipitating the starch solution, the starch solution can be heated to thereby produce cooked starch. For example, the starch solution can be heated to about 90 degrees Celsius. Precipitation of the starch solution can include: exchanging the water with ethanol under agitation to thereby form a starch precipitate; and filtering the starch precipitate. The resulting starch particles can be sized between 6 micrometers and 10 micrometers.

According to another aspect, under agitation and prior to the step of precipitating the starch solution, the starch solution can be heated to thereby produce cooked starch. Next, the step of precipitating the starch solution can comprise: combining the cooked starch with ethanol to thereby form a first emulsion; grinding the cooked starch in the first emulsion to thereby form a starch precipitate; and filtering the starch precipitate. The resulting starch particles can be sized between 3 micrometers and 8 micrometers.

According to another aspect, under agitation and prior to the step of precipitating the starch solution, the starch solution can be heated to thereby produce cooked starch. The step of precipitating the starch solution can comprise: combining the cooked starch with a nonionic surfactant and a solvent, such as, for example, toluene, to thereby form a first solution; agitating the first solution; combining the first solution with anhydrous ethanol to thereby form a starch precipitate; and filtering the starch precipitate. The starch precipitate can also be dried. The starch particles can be sized between 430 nanometers and 4.8 micrometers. Voids in the predefined void structure can have a diameter of about 140 nanometers.

According to another aspect, the starch can be gelatinized in the starch solution by combining the starch solution with an alkali such as sodium hydroxide. Further, the step of precipitating the starch solution can comprise: exchanging the water with ethanol under agitation to thereby form a starch precipitate; and filtering the starch precipitate. Voids in the predefined void structure can have a diameter of about 30 to 90 nanometers. The starch particles can be sized between about 5.0 micrometers and 10.2 micrometers.

According to another aspect, the starch can be gelatinized in the starch solution by combining the starch solution with an alkali such as sodium hydroxide. Further, the step of precipitating the starch solution can comprise: combining the gelatinized starch with a nonionic surfactant and a solvent to thereby form a first solution; agitating the first solution; combining the first solution with anhydrous ethanol to thereby form a starch precipitate; and filtering the starch precipitate. The starch particles can be sized between about 600 nanometers and 5.7 micrometers.

According to another aspect, the starch can be gelatinized in the starch solution by combining the starch solution with an alkali such as sodium hydroxide. Further, the step of precipitating the starch solution can comprise: combining the gelatinized starch with a nonionic surfactant and a solvent to thereby form a first solution; agitating the first solution; combining the first solution with anhydrous ethanol to thereby form a starch precipitate; neutralizing the first solution with an acid such as HCl; and filtering the starch precipitate. The starch particles can be sized between about 390 nanometers and 3.6 micrometers.

According to another aspect, a porous, starch-based foam structure is provided.

### BRIEF DESCRIPTION OF THE DRAWINGS

The foregoing summary, as well as the following detailed description of various embodiments, is better understood when read in conjunction with the appended drawings. For the purposes of illustration, there is shown in the drawings exemplary embodiments; however, the presently disclosed subject matter is not limited to the specific methods and instrumentalities disclosed. In the drawings:

FIG. 1 depicts a flowchart representing a method for producing a porous, starch-based foam structure according to one or more embodiments of the disclosed subject matter;

FIG. 2 depicts a flowchart representing a method for producing a porous, starch-based foam structure according to one or more embodiments of the disclosed subject matter;

FIG. 3 depicts a flowchart representing a method for producing a porous, starch-based foam structure according to one or more embodiments of the disclosed subject matter;

FIGS. 4(a) and 4(b) are Field Emission Scanning Electron Microscopy (FESEM) images of cooked Starch Micro-Cellular Foam (SMCF) particles using mechanical stirring to induce the precipitation process at two different magnifications: 0.5 K, as shown in FIG. 4(a); and 25 K, as shown in FIG. 4(b);

FIGS. 5(a) and 5(b) are FESEM images of cooked starch SMCF particles using the ball mill technique during the solvent exchange process at two different magnifications: 0.5 K, as shown in FIG. 5(a); and 50 K, as shown in FIG. 5(b);

FIGS. 6(a) and 6(b) are Scanning Electron Microscopy (SEM) images of cooked Starch Microcellular Foam (SMCF) particles using micro-emulsion technique at two different magnifications: 0.1, as shown in FIG. 6(a); and 15 K, as shown in FIG. 6(b);

FIGS. 7(a) and 7(b) are FESEM images of NaOH-gelatinized starch SMCF particles using mechanical stirring in the precipitation process at two different magnifications: 0.5 K, as shown in FIG. 7(a); and 50 K, as shown in FIG. 7(b);

FIGS. 8(a) and 8(b) are FESEM images of NaOH-gelatinized starch SMCF particles using the micro-emulsion technique at different magnifications: 1K, as shown in FIG. 8(a); and 75 K, as shown in FIG. 8(b);

FIGS. 9(a) and 9(b) are FESEM images of gelatinized starch SMCF particles using the micro-emulsion technique after neutralization of NaOH-with HCl at two different magnifications: 0.5 K, as shown in FIG. 9(a); and 30 K, as shown in FIG. 9(b); and

FIGS. 10(a) and 10(b) are FESEM images of neutralized SMCF particles stirred for one hour at 2000 revolutions per minute at two different magnifications: 5 K, as shown in FIG. 10(a); and 20 K, as shown in FIG. 10(b).

### DETAILED DESCRIPTION

The presently disclosed subject matter is described with specificity to meet statutory requirements. However, the description itself is not intended to limit the scope of this patent. Rather, the inventors have contemplated that the claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

Porous carbohydrate-based foam structures, such as those based on starch, as disclosed herein can be produced by precipitating, for example, a starch solution to form starch nanoparticles having a predefined void structure. The physical and chemical properties of the presently disclosed foam structures can be assessed to develop compounds based on natural starch polymers, which are readily available as a renewable natural polymer. These natural bio-compounds can be used as viable alternative products to synthetic petroleum based compounds with enhanced and improved properties. Additionally, the structures disclosed herein are also applicable to water soluble synthetic polymers.

In accordance with the presently disclosed subject matter, novel foam structures or compounds can be produced from water soluble polymers and inexpensive natural bio materials, including but not limited to, starch, water soluble cellulose, chitosan, sodium alginate, hemicelluloses, proteins, polyvinyl alcohols, and, in some instances, synthetic materials such as water soluble synthetic polymers. Various experiments are presented herein in which each of those experiments references a starch or starch solution. However, it is envisioned that each of the experiments may be carried out with the use of water soluble cellulose, chitosan, sodium alginate, hemicelluloses, proteins, polyvinyl alcohols, and all water soluble synthetic polymers, and other materials that may be commonly employed in the art. The compounds can have applications in several fields of engineering including civil, for example, for the use of light weight construction materials and composites, electrical, for example, for the use of materials having increased specific surface area and high opacity and brightness, agriculture, for example, for the use of host materials for release of suitable compounds, waste water treatment, paper, textiles, packaging, coating and paint industry, for example, as a filler material, as well as medical and pharmaceutical applications, for example, as a host material for the release of medical materials or as a bone replacement composite.

As referred to herein, starch is a high molecular weight polymer of anhydroglucose units (C₆H₁₀O₅) linked by alpha-D-glycosidic bonds. Starch, the principle carbohydrate storage biopolymer of plants, is an abundant, low cost resource. Starch is synthesized in plant cells as a food reserve. Starch can range from purely linear to extensively branched structures. As the granules populate, the molecules have the potential to form glycosidic conformations characteristic of helices or crystalline order. Starch is composed of two major molecules, amylose and amylopectin. Amylose is defined as a linear molecule but it is recognized that some molecules are slightly branched by (1-6)- [symbol] -linkages. Amylopectin is a branched polysaccharide constructed from hundreds of short (1-4)- [symbol] -glucan chains, which are interlinked by (1-6)- [symbol] -linkages. Amylopectin is primarily responsible for the granule structure and crystallinity in most starches.

Chemical modification of starch is an interesting approach to changing its chemical and physical properties. Such modifications can potentially open the way to increase the industrial applications of starch. Unmodified starch cannot satisfactorily replace the functional and physical properties of conventional plastics since starch-based plastics exhibit water sensitivity and weak mechanical characteristics. The mechanical properties of starch materials depend, to a great extent, on the amylose/amylopectin ratio. However, the thermoplastic starch can be an interesting alternative for synthetic polymers where long-term durability is not required and a rapid disintegration is an advantage.

Hydrophobic grafted starch nanoparticles can be produced by graft copolymerization of fatty acid on starch nanoparticles using an appropriate catalyst, such as potassium persulfate. This reaction is temperature and time dependent.

Selective modification of starch using enzymes may be beneficial compared with prior methods since the use of enzymes may have a low impact on the environment. One of the main challenges that face the regioselective modification of starch is the low degree of substitution. This is due to the use of polar aprotic solvents that strip critical water from enzyme thus lowering their activities and heterogeneous reaction conditions that restrict the modification of large particles and films to small fraction of the substrate residing at the surface. Enzymes can be used as catalyst during an esterification reaction. The enzyme is incorporated within reverse micelles using the anionic surfactant aerosol-OT [AOT, bis(2-ethylhexyl) sodium sulfosuccinate]. The AOT can form thermodynamic water droplets surrounded by a surfactant monolayer in oil, such as, for example, isooctane. Water entrapped within the reverse micelles resembles the polar pockets in cell. This increases the dissolution of the entrapped enzyme within the reverse micelles in nonpolar media and consequently facilitates productive collisions and reactions between enzymes and nonpolar substrate. The starch nanoparticles can be esterified using Candida Antartica Lipase B (CAL-B) as a catalyst in both its immobilized (Novozym 435) and free (SP-525) forms. The starch particles can be acylated by formation of Aerosol-OT [AOT, bis(2-ethylhexyl) sodium sulfosuccinate] stabilized micro-emulsions. Starch nanoparticles can be reacted with vinyl stearate, caprolactone, and maleic acid at 40 degrees Celsius for 48 hours with a degree of substitution of 0.8, 0.6, and 0.4, respectively. Using Integral Field (IF) spectroscopy, the substitution at the C-6 position of the repeating glucose units can be confirmed.

As referred to herein, precipitation is a process by which a solid is formed in a solution or inside another solid during a chemical reaction or by diffusion in a solid. When the reaction occurs in a liquid, the solid formed is known as the precipitate, and the liquid remaining above the solid is known as the supernate.

As referred to herein, microemulsions can be clear, stable, isotropic, liquid mixtures of oil, water and surfactant. The aqueous phase may contain salt(s) and/or other ingredients, and the oil may be a complex mixture of different hydrocarbons and olefins. In contrast to commonly-known emulsions, microemulsions can form upon simple mixing of the components and do not require the high shear conditions generally used in the formation of ordinary emulsions. Two basic types of microemulsions include direct such as oil dispersed in water and reversed such as water dispersed in oil.

As referred to herein, TRITON X-100® (C₁₄H₂₂O(C₂H₄O)ₙ) is a nonionic surfactant which has a hydrophilic polyethylene oxide group and a hydrocarbon lipophilic or hydrophobic group.

As referred to herein, non-polar organic solvent such as toluene, also known as methylbenzene, or toluol, is a clear water-insoluble liquid. It is an aromatic hydrocarbon used as a solvent. Other organic solvents may also be employed with the embodiments described herein.

As referred to herein, nanoparticles can be solid, colloidal particles which can consist of macromolecular substances that vary in size from one to 1,000 nanometers. Depending on the method of preparation, nanoparticles can be obtained with different properties. Nanoparticles can be used as host compounds and their release characteristics for the encapsulated therapeutic agents can be regulated based on the production method.

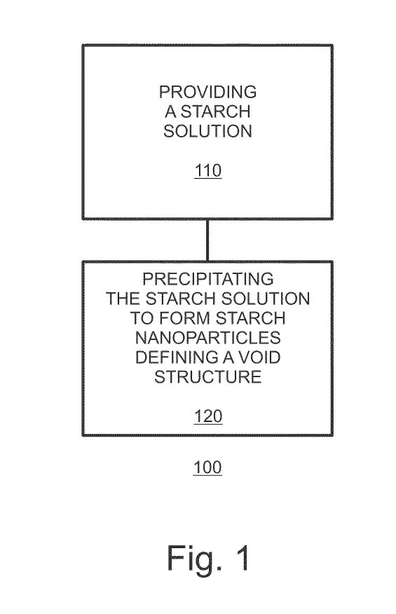

Methods and processes for producing a porous, starch-based foam structure are disclosed herein. A method 100 for producing a porous, starch-based foam structure is depicted in the flowchart of FIG. 1. The method 100 includes providing a starch solution 110 and precipitating the starch solution to form starch nanoparticles that define a void structure.

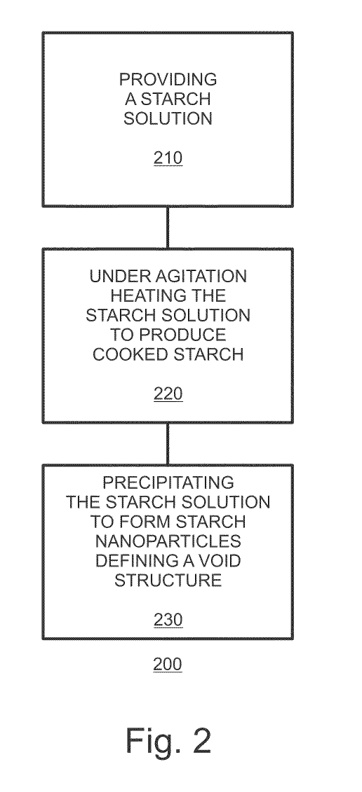

A method 200 for producing a porous, starch-based foam structure according to one or more embodiments of the disclosed subject matter is depicted in the flowchart of FIG. 2. The method 200 includes providing a starch solution 210, under agitation, heating the starch solution to produce a cooked starch 220, and precipitating the starch solution to form starch nanoparticles that define a void structure 230.

Agitation of the starch solution may be effectuated by any manner known in the art, and in one or more embodiments, may include using a mechanical or magnetic stirrer, using a ball mill, and stirring or agitating at varying speeds, intensities, and frequencies. Other manners of agitating the starch solution are described herein. Heating the starch solution 220 may also be effectuated by introducing the solution to heat in a variety of manners described herein.

Precipitating the starch solution 230 may be effectuated by, for example, exchanging water of the starch solution with a solvent having a lower surface tension than the water, and may further include repeating the exchanging step. Precipitating the starch solution 230 may also be effectuated by, for example, exchanging water of the starch solution with ethanol under agitation and filtering the starch precipitate. Precipitating the starch solution 230 may also be effectuated by, for example, combining a cooked starch with anhydrous ethanol, grinding the cooked starch to form a starch precipitate, and filtering the starch precipitate. Precipitating the starch solution 230 may also be effectuated by combining a cooked starch with a nonionic surfactant and a solvent to form a first emulsion, agitating the first emulsion, combining the first emulsion with anhydrous ethanol to form a starch precipitate, and filtering the starch precipitate. Other manners of precipitating the starch solution 230 are described herein.

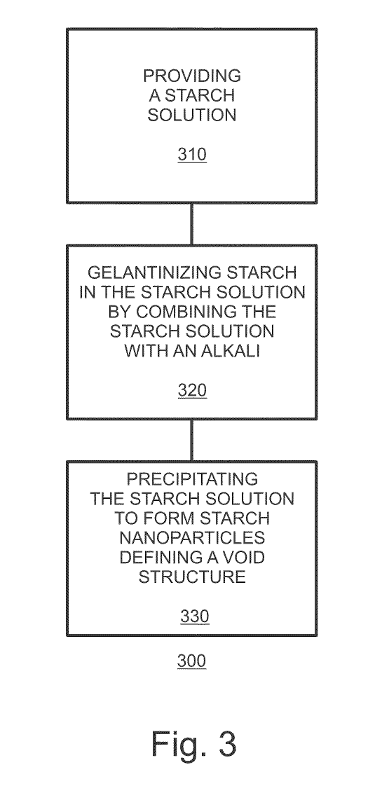

A method 300 for producing a porous, starch-based foam structure according to one or more embodiments of the disclosed subject matter is depicted in the flowchart of FIG. 3. The method 300 includes providing a starch solution 310, gelatinizing starch in the starch solution by combining the starch solution with an alkali 320, and precipitating the starch solution to form starch nanoparticles that define a void structure 330.

Gelatinizing starch in the starch solution by combining the starch solution 310 may be effectuated by, for example, combining the starch solution with an alkali such as sodium hydroxide. Other manners of gelatinizing starch in the starch solution 310 are described herein.

Precipitating the starch solution to form starch nanoparticles that define a void structure 330 may be effectuated by, for example, exchanging water of the starch solution with ethanol under agitation and filtering the starch precipitate. Precipitating the starch solution 330 may also be effectuated by, for example, combining the gelatinized starch with a nonionic surfactant and a solvent to form a first emulsion, agitating the first emulsion, combining the first emulsion with anhydrous ethanol to form a starch precipitate, and filtering the starch precipitate. Precipitating the starch solution 330 may also be effectuated by, for example, combining the gelatinized starch with a nonionic surfactant and a solvent to form a first emulsion, agitating the first emulsion, combining the first emulsion with anhydrous ethanol to form a starch precipitate, neutralizing the first emulsion with an acid such as HCL, and filtering the starch precipitate. Other manners of precipitating the starch solution 330 are described herein.

Starch nanoparticles may be prepared using a water/oil micro-emulsion technique. In this technique, starch solution can be mixed with a non-polar organic solvent in the presence of a surfactant to form one phase micro-emulsion solution. The starch nanoparticles can be prepared by crosslinking starch with epichlorohydrin in the presence of n-hexane, and a nonionic surfactant. By increasing the concentration of starch, the diameter of particles can be increased while broadening the distribution of particle size. The range of nanoparticles' diameter can be between 15.6 nanometers to 226 nanometers.

Polymers of foam structures have unique physical, mechanical and thermal properties. The properties of foams may be characterized by cell density, expansion ratio, cell size distribution, open cell content, and cell integrity. Different polymers with different properties will form foams having wide varieties of characteristics ranging from soft to stiff modulus, resilient to tough behavior, low to high hysteresis, and mono to multi-model cell distribution.

Several techniques may be used to produce foam structures. As a non-limiting example thereof, techniques may include freeze-drying, supercritical fluid extrusion, for example using supercritical carbon dioxide, and solvent exchange technique. Each of these techniques is described herein below.

Freeze-drying is the process of dehydrating starch and its derivatives under a vacuum, where the moisture content changes directly from a solid to a gaseous form through sublimation without having to undergo the intermediate liquid state. In this process, the product maintains its original size and shape with a minimum cell rupture. Removing moisture prevents the product from deteriorating at room temperature. The freeze drying technique may be used to produce starch microcellular foam with cell size less than 100 micrometers. In this approach, cooked starch is poured in molds and cooled in the refrigerator, and a semi-rigid structure is obtained. The cooked starch is then frozen, during which the water forms ice crystals and is separated from the solute. Freeze drying of the frozen starch sublimates the ice crystals and forms a porous starch cake structure.

The physical properties of gelatinized tapioca starch beads, including, structure, pore size and porosity, may be modified by freeze drying at different temperatures. The bacterial immobilization in freeze-dried tapioca beads may be a simple technique for enhancing delivery of viable probiotics culture to the intestinal tract. Edible bilayer film may be used to coat the dried immobilized beads as an additional protective layer.

The thermal conductivities of freeze-dried slabs of starch, gelatin, pectin, cellulose gum, and egg albumen gels have been determined under a variety of conditions, using a guarded hotplate apparatus. The thermal conductivities of dry materials at atmospheric pressure vary from 0.921×10⁻⁴ calories centimeters⁻¹ degrees Celsius⁻¹ seconds⁻¹ (gelatin) to 1.337×10⁻⁴ for materials such as cellulose gum. In a vacuum, this variation is from 0.218×10⁻⁴ for materials such as starch to 0.467×10⁻⁴ calories centimeters⁻¹ degrees Celsius⁻¹ seconds⁻¹ for materials such as cellulose gum. The difference between atmospheric pressure and vacuum is equal to the thermal conductivity of air for all the materials except cellulose gum, which gives a greater difference. The thermal conductivity of starch gel increases linearly with increasing temperature from zero to 70 degrees Celsius, and decreases with decreasing pressure, as porous materials normally do, to a constant value below 0.1 millimeters of mercury. A helium atmosphere gives a higher thermal conductivity than air or nitrogen. The thermal conductivity is higher in all freeze-dried gels containing adsorbed water than after the removal of all the water. The thermal conductivity of pectin gels increases with the density. Thermal conductivity is also affected by the type and size of pores of the dried materials. In general, changes in thermal conductivity are significant with pressure, type of gas, and nature of the material, particularly the fibrous structure, but less important with temperature and amount of adsorbed water.

Supercritical fluid extrusion (SCFX) is an important field of research in the extrusion industry. This technique has been used for the production of highly expanded biopolymeric foams, such as starch-based polymers, in which carbon dioxide, above its critical pressure of 7.38 megapascals and temperature of 31 degrees Celsius, is used as a blowing agent, a nutrient carrier, and an in-line process modifier. The use of supercritical carbon dioxide (SC—CO₂) leads to avoiding the use of steam, shear reduction, and enablement of the use of low production temperatures, such as less than 100 degrees Celsius. These, in turn, offer several advantages over the conventional method of steam puffing including reduced product degradation, decreased machine wear, and the potential for adding heat sensitive nutrients, flavors, and colorants in-line. Moreover, in starch microcellular foam processing, the subtle phase change of injected CO₂ from supercritical to gas is exploited to obtain precisely controlled, homogenous, and closed cell microcellular structures and unique product textures. This represents an improvement over puffed food products produced by conventional extrusion where the more explosive phase change from water to steam leads to very coarse and open cell structures. The SCFX process for starch-based foams may include development of a melt with gas holding rheological properties by gelatinization and mixing of the feed in the extruder barrel, injection of SC—CO₂ loaded with solutes, if desired, into the melt and mixing in the extruder barrel to create a melt-CO₂ solution, pressure drop in the extruder nozzle leading to a thermodynamic instability and consequent nucleation of bubbles, and expansion caused by diffusion of CO₂ into the nucleated bubbles as the extrudate proceeds through the nozzle and immediately after its exit from the nozzle. In the final stage, after the nascent foam exits the nozzle, further diffusion of CO₂ into the bubbles and their consequent expansion cause the bubble walls to decrease progressively in thickness, and increase the rate at which CO₂ diffuses out of the foam to the environment. This causes the amount of CO₂ available for bubble growth to decrease and could even lead to foam collapse as observed in SCFX processing, which also has been reported for polymeric foams. By controlling the effective diffusivity of CO₂ through the starch-based foam, potential exists for controlling the cellular structure and increasing product expansion, thus manipulating the mechanical and textural properties of the end product.

Starch microcellular foam has been successfully produced with a bubble size in the range of 50 to 200 microns, and bubble density to the order of 10⁶ per cm³. The bubble size and expansion ratio of extrudates depends on process and material parameters including CO₂ injection rate, nozzle temperature, oven temperature, melt viscosity, melt yield stress, etc.

Production of expanded starch-based foams with microcellular structure can be achieved by injection of supercritical CO₂ into the melt. The high effective diffusivity of CO₂ in the porous matrix may allow for escape of the gas to the environment, reducing the amount available for diffusion into the bubbles, thus posing an important challenge. Several approaches may address this problem such as increasing the nucleation rate and thus the final bubble density in the foam, and reducing the melt temperature. Increasing the nucleation rate may be achieved by decreasing the nozzle diameter in order to achieve a higher pressure drop rate as the starch-CO₂ melt flows through the nozzle. A cooling zone may be used prior to the entry of the melt into the nozzle to assess the feasibility of the second approach. Bubble density can increase more than fourfold when the nozzle radius is decreased from 3.00 to 1.50 millimeters. Higher bubble density can lead to a greater barrier or resistance to diffusion of CO₂ to the environment, and increases expansion ratio by as much as 160 percent. Cooling of the melt also results in a decrease in diffusion coefficient of CO₂ in the starch melt, and thus reduces CO₂ loss to the environment. The expansion ratio is thus increased by 34 percent as the melt temperature is decreased from 60 to 40 degrees Celsius.

Starch Microcellular Foam (SMCF) compounds are also disclosed herein and may be produced using a solvent exchange technique from native corn starch crosslinked with glutaraldehyde. In order to produce starch particles with a high specific surface area, the cooked starch solution can be reacted with glutaraldehyde and then precipitated under shear in presence of ethanol. The solvent is exchanged another two times under continuous stirring such as, for example, 2200 revolutions per minute. Nuclear Magnetic Resonance technique can be used to confirm the crosslinking reaction. It has been found that an increase in the glutaraldehyde concentration from zero to 15 grams per 100 grams starch is accompanied by a decrease in particle size, moisture content, brightness, and specific surface area. Scanning electron microscope (SEM) images of the SMCF particles show that the smallest average void diameter obtained was 0.182 micrometers at a 7.5 grams glutaraldehyde per 100 grams of starch. In an experiment, four starch compounds with different viscosities were prepared by hydrolysis of the native corn starch with a mixture of HCl and methanol (1 N HCl/methanol) system for different durations. One purpose of this experiment was to investigate the relationship between starch molecular weight and the resultant void structure of the SMCF. The starch compounds were crosslinked with 15 grams of glutaraldehyde per 100 grams of starch and precipitated with ethanol to form SMCF. Decreasing the starch viscosity decreased the brightness and specific surface area, whereas, particle size, void diameter and moisture content increased. Increases in stirring speed during the precipitation enhanced the precipitation process of the SMCF particles. Pressing the starch particles to form a pellet caused a collapse of the foam structure at pressures above about 6000 pounds per square inch.

Biodegradable polymers such as native starch and polylactic acid (PLA) may have commercial applications, such as for packaging materials. Their hydrophilic properties, however, hinder their widespread use in various applications. Hydrophobicity can be improved significantly by acetylating native starch. Acetylated corn starch, with a degree of substitution (DS) 2.3, and acetylated potato starch, with a DS 1.07, may be extruded with 5, 10, or 15 percent PLA in a twin screw co-rotating extruder at 150, 160, or 170 degrees Celsius barrel temperature and 130, 150, or 170 revolutions per minute screw speeds. Analytical techniques including DSC, XRD, and FTIR spectroscopy may be used to analyze the morphological properties of the extruded foams. One of ordinary skill may be aware that a central composite response surface design can be used to analyze the effects of acetylated starch type, PLA content, barrel temperature and screw speed on the specific mechanical energy requirements of preparing extruded foams and the radial expansion ratios and compressibility of the extruded foams. In one experiment, test data showed that the glass transition temperature (Tg) and melting temperature (Tm) of corn starch-PLA foams (DS 2.3) were between the Tg and Tm of corn starch and PLA. The Tg's of potato starch-PLA foams (DS 1.07) were higher than those of acetylated potato starch and PLA, while the Tm's were closer to that of the PLA, when acetylated potato starch was the predominant phase in the blends. XRD showed that both acetylated starch and PLA lost crystallinity during extrusion. The X-ray pattern of the DS 1.07 potato starch-PLA foam was similar to that of potato starch and PLA (DS 1.07). FTIR spectroscopy confirmed no new bonds were formed in either DS 2.3 corn starch-PLA or DS 1.07 potato starch acetate-PLA foams. The type of acetylated starch, PLA content, barrel temperature, and screw speed had significant effects on the specific mechanical energy requirements, radial expansion ratios, and compressibility of the acetylated starch foams.

Characterization of the functional proprieties of extruded starch acetate foams based on the morphological properties after extrusion transformation may be an important tool to obtain the optimum functional properties of such packaging materials. Corn starch acetate with a degree of substitution (DS) of 2.30 and potato starch acetate with a DS of 1.09 can be extruded with 10 percent, 15 percent, and 20 percent ethanol in a twin screw extruder using screw speeds of 110, 130, and 150 revolutions per minute and barrel temperatures of 130, 150 and 170 degrees Celsius. A response surface design can be applied to analyze the effects of ethanol content, screw speed, and barrel temperature on the physical properties, mechanical properties, and macromolecular properties of the extruded starch acetate foams. It has been shown that ethanol content, barrel temperature and screw speed had significant effects on the functional properties of extruded starch acetate foams. Because of the differences in molecular structure degradation in the corn and potato starch acetates, the functional properties of extruded corn starch acetate foams were higher than those of extruded potato starch foams. This was substantiated based on the significant macromolecular structural differences between the two varieties.

Biodegradable food service packaging including a starch-fiber core and a biodegradable film laminate has been produced. The biodegradable films were made of polylactic acid (PLA), polybutylene succinate/terephthalate (PBST), rubber latex and polybutylene adipate/terephthalate (PBAT). This technique involves an in situ process for laminating a baked foam product in a single step. The in situ technique involves a critical element of using a heat insulating fiber sheet to stabilize heat-sensitive laminate films during the baking/lamination process. The PLA-, PBST- and PBAT-laminated samples thus prepared were baked for six minutes at 120 degrees Celsius. The latex-laminated sample, which is more heat-stable, does not need to be heat-insulated and can be baked for three minutes at 160 degrees Celsius. Starch-based foam laminated with PLA, PBST or PBAT generally has higher density and greater tensile and flexural strength than the non-laminated counterpart. Starch foam laminated with a rubber latex film was shown to have tensile and flexural properties similar to the non-laminated sample, due to the low modulus and elasticity of the latex film. The in situ lamination process may improve the adhesion of the starch foam core with the fiber sheet, PLA and latex films compared to a post-lamination process. This process may also indicate that all of the laminated materials provided a low water vapor permeance. A comparison of the degradation of treated and unlaminated films in a compost mixture showed that laminated films degraded at a much slower rate compared to native starch.

Different types of starch may be used for producing a porous, starch-based foam structure in accordance with the presently disclosed subject matter. For example, native corn cooked starch and pre-gelatinized starch may be used.

Native cooked starch can be produced by subjecting a starch slurry to continuous stirring and heating at a rate of (1 degree Celsius per minute) to 95 degrees Celsius and keeping it at that temperature for 30 minutes. Starch nanoparticles can be produced through subsequent steps.

Pregelatinized starch can be produced by treating Starch slurry with 2 to 5 percent sodium hydroxide solution at room temperature for 30 minutes under continuous stirring. The pH of starch slurry can then be adjusted between four and eight based on the type of crosslinking agents used, which in one or more embodiments, may be epichlorohydrin, phosphorous, or oxychloride. Different concentrations of the crosslinking agent can be used to determine the optimum conditions.

Crosslinked starch nanoparticles may be produced using a technique employing a water/oil micro-emulsion. Starch micro-emulsion can be prepared in a three necked flask with a mechanical stirrer, condenser, and rubber septum. The preparation technique can be adjusted with 50 milliliter of n-hexane/toluene/isooctane, 10 milliliter of a surfactant such as sorbitan monooleate (Span-80/bis) (2-ethylhexyl) sulfosuccinate (AOT)/TRITON X-100® (polyoxyethylene-10-isooctylphenyl ether), and a crosslinking agent and aqueous starch solution such as, for example, 20 grams of 10 starch solution. The emulsion can then be subjected to a high shear rate such as, for example, 1000, 2000, 3000, 5000 or 7000 revolutions per minute for 30 minutes. Different parameters can affect particle size formation and extent of crosslinking, such as ratio of water in oil, surfactant concentration, reaction temperature, duration and shear rate.

Starch nanoparticles may be precipitated using a solvent exchange technique. In this process, solvent of higher surface tension, such as, for example water, may be replaced with another solvent of lower surface tension, such as, for example ethanol, isopropanol, or acetone. This exchange leads to the formation of a foam structure. This process can be repeated to ensure all water molecules are exchanged with ethanol. Optimization of the precipitation process can be employed, since it can be a factor that controls the void structure of the nanoparticles. Finally, the starch nanoparticles can be dried at 50 degrees Celsius for four hours before characterization.

Two presently disclosed methods may be used to modify starch nanoparticles. These methods may include, for example, enzyme-catalyzed regioselective modification, and chemical catalytic modification.

In enzyme-catalyzed regioselective modification, starch nanoparticles can be chemically modified using enzyme as catalyst in nonaqueous medium in the presence of different acylating agents such as, but not limited to, maleic anhydride, butyric anhydride and Palmitic anhydride. Different conditions affecting the modifications process can optimize the esterification reaction. The modified nanoparticles can be filtered, purified and then dried at room temperature.

In chemical, catalytic modification, starch nanoparticles can be chemically modified in a non-aqueous medium, such as, for example dimethyl formamide or dimethyl acetamide, in the presence of different alkyl halides, such as, for example octanoyl chloride, benzoyl chloride and palmotyl chloride, as well as dimethylpyridine as a catalyst. The chemical modification can occur on the surface of the particles which in turn increases the hydrophobicity of the starch particles. Factors affecting the reaction efficiency include, but are not limited to, reaction temperature, acyl chloride concentration, catalyst type and concentration.

In a cooked starch preparation experiment, a solution of 20 percent starch was prepared by introducing 20 grams of starch into a three-necked round flask containing 100 milliliter of distilled water. A mechanical stirrer, septum and condenser were then fixed and the flask was placed in an oil bath. Under continuous stirring, the temperature was raised to 90 degrees Celsius and kept constant for 30 minutes. Finally, the solution containing 20 grams of starch was introduced to a three-necked flask as described below.

According to one experiment referred to herein as experiment A-1, water was exchanged with ethanol under continuous and vigorous stirring for ten minutes. The precipitate was filtered off and re-introduced into the flask. Another 75 milliliter of anhydrous ethanol was added and the starch paste was stirred at 2400 revolutions per minute for one hour. This step was repeated twice to eliminate all water molecules and ensure a complete exchange with ethanol.

According to one experiment referred to herein as A-2, a starch solution of 20 grams was placed inside a 50 milliliter iron cell containing 20 grinding balls. 30 milliliter of ethanol was added. The iron cell was closed and placed in a ball mill machine for 15 minutes. The starch particles were filtered off and placed again in the cell followed by the addition of 50 milliliter of ethanol and the grinding was continued for another 30 minutes. The last step was repeated twice. Finally, the starch was filtered off, washed and then dried at room temperature.

According to one experiment referred to herein as A-3, 20 grams of cooked starch was added to a three-necked flask containing 50 milliliter of toluene and 15 gram of TRITON X-100®. The solution was stirred for 30 minutes at 2000 revolutions per minute. The micro-emulsion was poured on 100 milliliter of anhydrous ethanol. Starch particles were precipitated immediately and then filtered-off. Final washing of starch particles with ethanol can be carried out using a magnetic stirrer for 30 minutes.

According to another experiment, starch particles were prepared using a solvent exchange technique in which the water molecules are exchanged with a lower surface tension solvent such as ethanol. During the precipitation process, a mechanical stirrer, a ball mill, and a micro-emulsion technique were used.

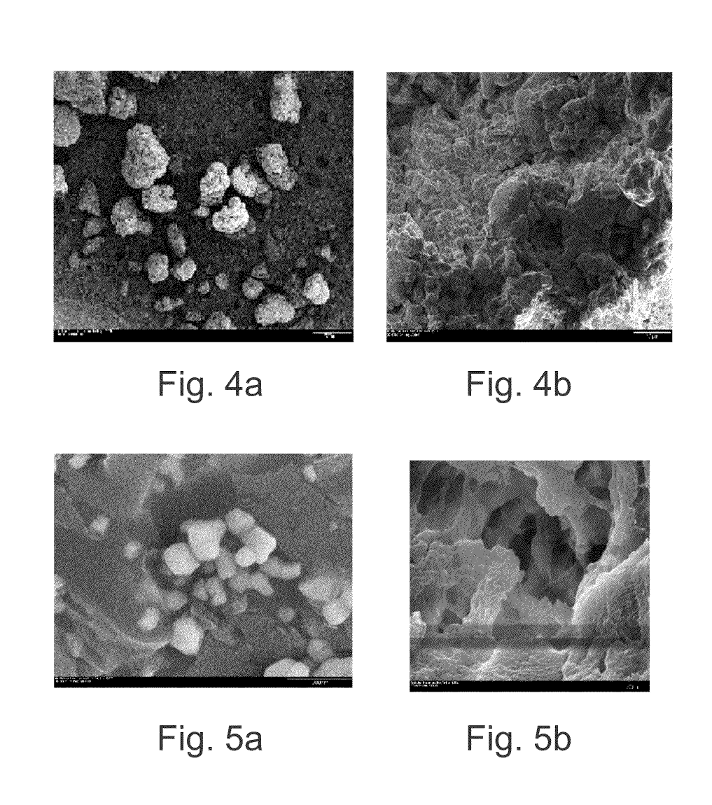

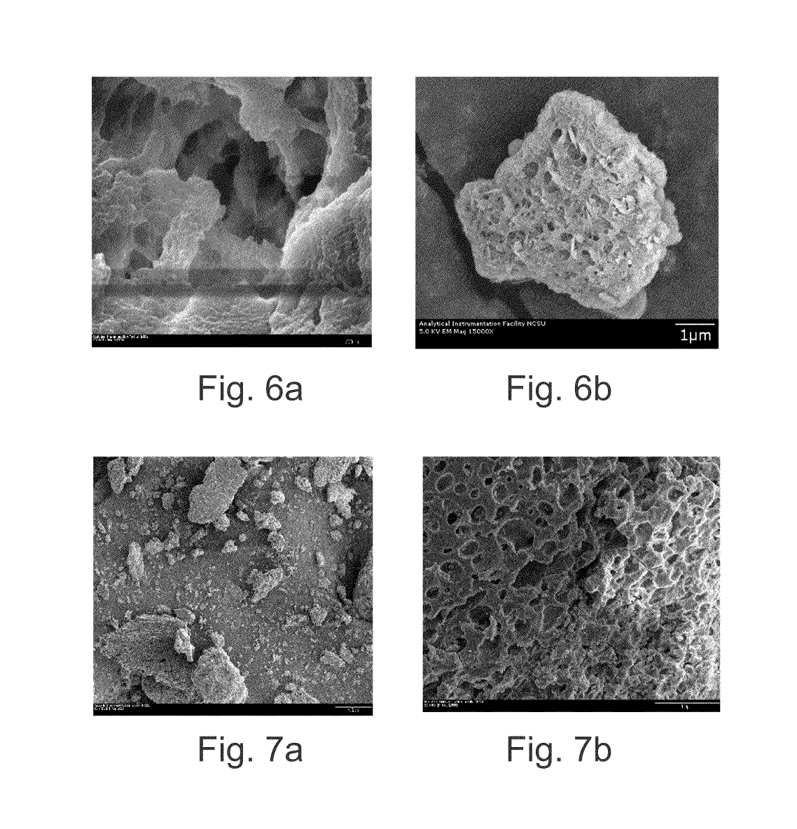

Field Emission Scanning Electron Microscopy (FESEM) was used to study the morphology of the foam particles obtained in the initial phase of the above-described experiments. All micrographs were recorded at a magnification range of 100 to 30000. In testing, it was observed that the particle size decreases with an increase in the molecular weight and stirring rate. Results of experiment A-1, described above, showed starch particles had an inconsistent void structure with an average particle size of 10.3 micrometers as shown in FIGS. 4(a) and 4(b). Seventy-five starch particles were measured to calculate the average particle size. FIGS. 5(a) and 5(b) show the FESEM micrographs of starch particles obtained from experiment A-2 using a ball mill to precipitate and grind the starch particles to finer particles. A clear foam structure was shown with a slight collapse in the void structure. A mixture of both starch nano- and micro-particles was obtained using the micro-emulsion technique described in experiment A-3. FIGS. 6(a) and (b) show a foam structure with a wide range of particle sizes from 430 nanometers to 4.8 micrometers with an average void diameter of 140 nanometers. The data indicated that the particle size of the SMCF was greatly affected by the technique applied during the solvent exchange precipitation process.

In a gelatinized starch experiment, under continuous stirring, 20 grams of starch was gelatinized by adding three to five percent of weight sodium hydroxide to an aqueous 20 percent slurry. The paste was divided into three equal parts and treated as described below.

In one experiment referred to as experiment B-1, the gelatinized starch was allowed to precipitate by mixing 50 milliliter of anhydrous ethanol under vigorous stirring for 10 minutes. Starch particles were filtered-off and washed with ethanol. The starch particles were then transferred to a flask, followed by addition of 75 milliliter ethanol and were stirred at 2400 revolutions per minute for one hour and then filtered. The last step was repeated twice under the same conditions. Finally, the solution was filtered and dried.

In one experiment referred to as experiment B-2, 20 grams of alkali gelatinized starch was added to a three-necked flask containing 50 milliliter of toluene and 15 grams of TRITON X-100®. The solution was stirred for 30 minutes at 2000 revolutions per minute. The micro-emulsion solution was poured on 100 milliliter of anhydrous ethanol. Starch particles were precipitated immediately and then filtered-off. Final washing of starch particles with ethanol was carried out using a magnetic stirrer for 30 minutes.

In one experiment referred to as experiment B-3, the gelatinized starch underwent the same procedure as that used in experiment B-2. However, the starch solution was neutralized with Hydrochloric acid (HCl).

Gelatinization of starch with sodium hydroxide was used to dissolve the starch in water. Such an experiment was carried out at room temperature as an alternative to cooking starch at a higher temperature for a longer period, such as, for example, 95 degrees Celsius for 30 minutes. The gelatinization is an economical way to dissolve starch in water.

FIGS. 7(a), 7(b), 8(a), 8(b), 9(a), and 9(b) show the surface characteristics of starch particles when stirring and micro-emulsion techniques were used to precipitate the starch particles during the solvent exchange technique to exchange the water molecules with ethanol. FESEM micrographs show that all the samples have a foam structure. Results reveal that on average higher particles sizes, 8 micrometers with one to three micrometers void diameter were obtained when the stirring technique was used to precipitate the starch particles as depicted in FIGS. 7(a) and 7(b).

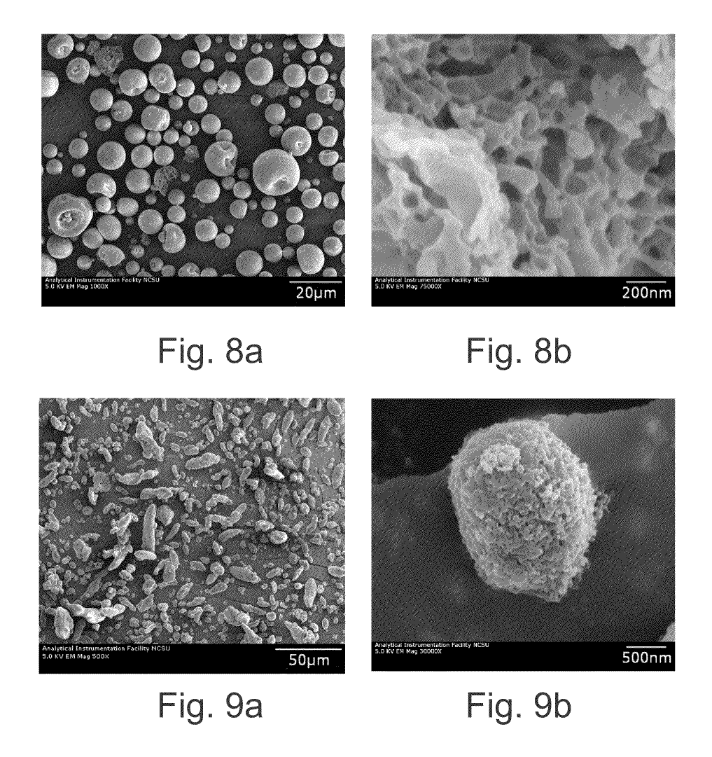

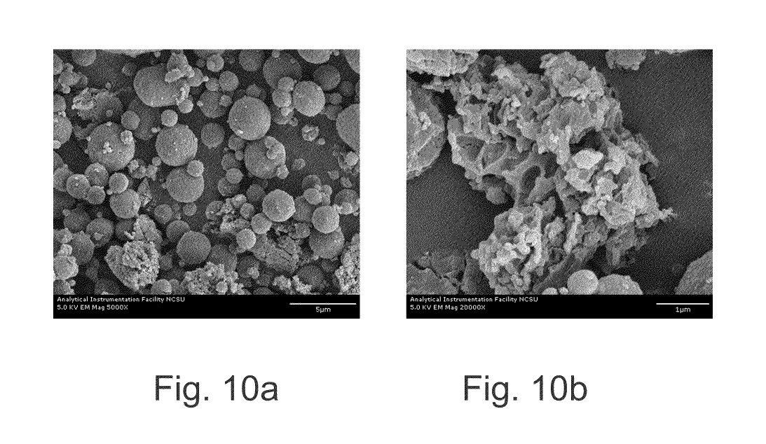

The use of the micro-emulsion technique to produce nanoparticles from gelatinized starch proved effective to obtain particles with a foam structure when alkaline and neutral starch solutions were used. The average void diameter for all samples was between about 30 and about 90 nanometers. Results also indicated a difference in the particle formation as shown in FIGS. 8(a), 8(b), 9(a) and 9(b) for the same emulsion solution. This data indicates that a successful synthesis of starch nanoparticles can be achieved with a wide range of particle sizes.

The effect of the precipitation process on the starch particle size is shown in Table 1 in which “N” indicates a neutral solution and “A” indicates an alkaline solution. Starch particle sizes were determined using software programs. The data indicated that gelatinized starch particles treated with five percent sodium hydroxide when precipitated according to the conditions discussed above had the smallest particle size of 4.17 micrometers.

TABLE 1
The Precipitation Process on Starch Particle Size

| | | | | Average |
|---|---|---|---|---|
| Type of | Experi- | | | Particle Size of |
| Starch | ment | Precipitation | Particle Size | 75 Particles |
| Treatment | Code | Process | (micrometers) | (micrometers) |
| Heat | (A-1) N | Stirring technique | 6-10 | 10.3 |
| Treated | (A-2) N | Ball mill | 3-8 | 7.15 |
| Starch | (A-3) N | Micro-emulsion | 0.430-4.8 | 3.00 |
| NaOH | (B-1) A | Stirring technique | 5.0-10.2 | 8.17 |
| Treated | (B-2) A | Micro-emulsion | 0.6-5.7 | 3.70 |
| Starch | (B-3) N | technique | 0.390-3.6 | 1.89 |

Transition electron microscopy (TEM) was used for particle size determination and to clarify the nanostructure of the starch particles. Images were recorded at 200 kilovolts in bright field mode along with the corresponding diffraction patterns. The size of the starch particles were measured for at least 150 particles. The TEM samples are prepared by placing the particles onto a carbon coated TEM copper grid.

Differential scanning calorimetry (DSC) with a manual liquid nitrogen cooling system was performed on sample materials disclosed herein. Starch powder, conditioned at zero percent relative humidity over P₂O₅ in desiccators, was placed in a hermetically closed DSC crimp-sealed pan. Samples were tested in a range from −100 to 350 degrees Celsius at a heating rate of 10 degrees Celsius minute⁻¹ under a nitrogen atmosphere.

Morphological characterization of starch nanocellular foams (SNCF) was carried out on images acquired using a scanning electron microscope (SEM), and the samples were deposited on a 10 nanometers thick platinum plate to make the samples conductive. The void diameters of SNCF apparent on the particle surface were determined using after spatial calibration using the length scale provided with the SEM micrograph. The apparent density values were also obtained.

The brightness of the SNCF powder was measured using a computing device and a brightness measurement device to measure the brightness of the SNCF as a function of chemical modification.

Photomicrograph pictures of SNCF were taken using a color video camera connected to a microscope. Commercially available software was used to measure the particle size.

The surface area of the SNCF was determined with a BET surface area analysis using 30 percent N₂ in Helium gas.

Fourier transform infrared (FTIR) spectroscopy was used to characterize native and modified starch. The samples were ground with potassium bromide (KBr) and pressed into pellets for FTIR transmission measurements.

High resolution solid-state NMR is another important tool was used to investigate the molecular structural changes of native and modified starch using a Spectrometer. The CP/MAS experiment may be conducted, for example, on a four millimeter MAS probe.

The moisture content of the SNCF was determined using, for example, thermal gravimetric analysis on samples exposed to ambient conditions. The samples were heated to 120 degrees Celsius for 30 minutes at a temperature rate increase of five degrees Celsius per minute.

Elemental analysis was performed using an elemental spectrometer. The sample was placed in a tin container, dropped in a furnace with oxygen at 950 degrees Celsius, and the carbon was measured as CO₂ in an infrared cell (IR cell) and the hydrogen was measured as water in an IR cell. The gasses were then swept over hot copper sticks to remove oxygen and reduce the NO₂ to N₂, the CO₂ and H₂O were removed and the N₂ measured by a thermal conductivity cell. The graft yield percent of the modified starch was calculated according to the following equation:

$$Add on percent = \frac{{C}_{ms} {C}_{st}}{{C}_{M} {C}_{ms}} \times 100$$

where: Cₘₛ is the percent carbon in the modified starch (X percent), Cₛₜ is the percent carbon in the dry native starch (Y percent), and C_{M} is the theoretical percent C of acylating agent.

Fourier transform infrared spectroscopy was used to characterize native and modified starch. The samples were ground with potassium bromide (KBr) and pressed into pellets for FTIR transmission measurements.

High resolution solid-state NMR was used to investigate the molecular structural changes of native and crosslinked starch using a spectrometer. The CP/MAS experiment was run on a 4 millimeter MAS probe.

While the embodiments have been described in connection with the particular embodiments of the various Figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

What is claimed:

1. A method for producing a porous, starch-based foam structure, the method comprising:
providing a starch solution; and
precipitating the starch solution to form starch nanoparticles having a predefined void structure.

2. The method according to claim 1, wherein the starch solution comprises a starch slurry of at least one of native corn cooked starch, wheat cooked starch, and potato cooked starch.

3. The method according to claim 1, wherein the starch solution comprises starch slurry of gelatinized starch.

4. The method according to claim 1, wherein the starch solution comprises water and starch, the starch particles comprising about 20% of the starch solid content.

5. The method according to claim 1, wherein the starch solution comprises water and starch particles, and wherein the step of precipitating the starch solution comprises:
exchanging the water with a solvent having a lower surface tension than the water; and
repeating the step of exchanging the water with a solvent having a lower surface tension than water.

6. The method according to claim 1, further comprising producing hydrophobic starch microcellular foam (SMCF) compounds by adding an alkyl ketene dimer (AKD).

7. A porous, starch-based foam structure produced in accordance to claim 1.

8. A method for producing a porous, starch-based foam structure, the method comprising:
providing a starch solution of water and starch;
under agitation, heating the starch solution to produce cooked starch; and
precipitating the starch solution to form starch nanoparticles that define a void structure.

9. The method according to claim 8,
wherein the step of precipitating the starch solution comprises:
exchanging the water with ethanol under agitation to form a starch precipitate; and
filtering the starch precipitate.

10. The method according to claim 9, wherein the starch nanoparticles are sized between 6 micrometers and 10 micrometers.

11. The method according to claim 8, wherein the step of precipitating the starch solution comprises:
combining the cooked starch with anhydrous ethanol;
grinding the cooked starch to form a starch precipitate; and
filtering the starch precipitate.

12. The method according to claim 9, wherein the starch nanoparticles are sized between 3 micrometers and 8 micrometers.

13. The method according to claim 8,
wherein the step of precipitating the starch solution comprises:
combining the cooked starch with a nonionic surfactant and a solvent to form a first emulsion;
agitating the first emulsion;
combining the first emulsion with anhydrous ethanol to form a starch precipitate; and
filtering the starch precipitate.

14. The method according to claim 13, wherein the starch nanoparticles are sized between 430 nanometers and 4.8 micrometers.

15. The method according to claim 13, wherein voids in the predefined void structure have a diameter of about 140 nanometers.

16. The method according to claim 13, wherein the solvent comprises toluene or other solvents.

17. The method according to claim 13, further comprising drying the starch precipitate.

18. The method according to claim 8, wherein the step of heating the starch comprises heating the starch solution to a temperature of about 90 degrees Celsius.

19. The method according to claim 8, further comprising chemically modifying nanoparticles to form starch nanoparticles comprising hydrophobic surfaces.

20. The method according to claim 19, wherein the surfaces of the starch nanoparticles have antimicrobial properties.

21. The method according to claim 20, wherein chemically modifying nanoparticles comprises graft copolymerization of fatty acid on the starch nanoparticles using a catalyst.

22. The method according to claim 21, wherein the catalyst is an enzyme.

23. The method according to claim 22, wherein chemically modifying nanoparticles comprises esterifying the starch nanoparticles using Candida Antartica Lipase B (CAL-B) as a catalyst.

24. The method according to claim 21, wherein chemically modifying nanoparticles comprises acylating the starch nanoparticles by formation of Aerosol-OT [AOT, bis(2-ethylhexyl) sodium sulfosuccinate] stabilized micro-emulsions.

25. A porous, starch-based foam structure produced in accordance to claim 8.

26. A method for producing a porous, starch-based foam structure, the method comprising:
providing a starch solution of starch and water;
gelatinizing starch in the starch solution by combing the starch solution with an alkali; and
precipitating the starch solution to form starch nanoparticles that define a void structure.

27. The method according to claim 26, wherein the step of precipitating the starch solution comprises:
exchanging the water with ethanol under agitation to form a starch precipitate; and
filtering the starch precipitate.

28. The method according to claim 27, wherein voids in the predefined void structure have a diameter of about 30-90 nanometers, and wherein the starch nanoparticles are sized between about 5.0 micrometers and 10.2 micrometers.

29. The method according to claim 26, wherein the step of precipitating the starch solution comprises:
combining the gelatinized starch with a nonionic surfactant and a solvent to form a first emulsion;
agitating the first emulsion;
combining the first emulsion with anhydrous ethanol to form a starch precipitate; and
filtering the starch precipitate.

30. The method according to claim 29, wherein the starch nanoparticles are sized between about 600 nanometers and 5.7 micrometers.

31. The method according to claim 26, wherein the step of precipitating the starch solution comprises:
combining the gelatinized starch with a nonionic surfactant and a solvent to form a first emulsion;
agitating the first emulsion;
combining the first emulsion with anhydrous ethanol to form a starch precipitate;
neutralizing the first emulsion with an acid such as HCl; and
filtering the starch precipitate.

32. The method according to claim 31, wherein the starch nanoparticles are sized between about 390 nanometers and 3.6 micrometers.

33. A porous, starch-based foam structure produced in accordance to claim 26.

34. A method for producing a porous foam structure, the method comprising:
providing an organic solution; and
precipitating the organic solution to form nanoparticles having a predefined void structure.

35. The method according to claim 34, wherein the organic solution comprises a starch slurry of at least one of native corn cooked starch, wheat cooked starch, and potato cooked starch.

36. The method according to claim 34, wherein the organic solution comprises starch slurry of gelatinized starch.

37. The method according to claim 34, wherein the organic solution comprises water and starch particles, and wherein the step of precipitating the organic solution comprises:
exchanging the water with a solvent having a lower surface tension than the water; and
repeating the step of exchanging the water with a solvent having a lower surface tension than water.

38. A porous foam structure produced in accordance to claim 34.
